(19) United States
(12) Patent Application Publication
(10) Pub. No.: US 2006/0235298 A1
(43) Pub. Date: Oct. 19, 2006

(54) Internal biopsy marking

(76) Inventors: Robert Kotmel, Burlingame, CA (US); Russel M. Sampson, Palo Alto, CA (US); Ellen Sheets, Concord, MA (US)

Correspondence Address: FISH & RICHARDSON P.C., PO BOX 1022, MINNEAPOLIS, MN, 55440-1022, US

(21) Appl. No.: 11/394,146

(22) Filed: Mar. 29, 2006

Related U.S. Application Data
(60) Provisional application No. 60/667,390, filed on Mar. 31, 2005.

(51) Int. Cl. A61B 6/00

(52) U.S. Cl. 600/431

(57) ABSTRACT

A system and method for marking a tissue site are described. A marker is deployed into or near the tissue site, where the marker can be later located using an imaging device external to the patient's body. After withdrawing the internal imaging device, the location of the marker can be identified using an external imaging device. Optionally, a biopsy of the internal tissue site can be performed based on the location of the marker.

### CROSS REFERENCE TO RELATED APPLICATION

This application claims priority to pending U.S. Provisional Application Ser. No. 60/667,390, entitled “Intraductal Biopsy Marking” , filed on Mar. 31, 2005, the entire contents of which are hereby incorporated by reference.

### TECHNICAL FIELD

This invention relates to a system and method for marking a tissue site.

### BACKGROUND

A minimally invasive procedure can be used to explore tissue within a patient's body to search for suspected unhealthy tissue, for example, cancer cells. One example of such a minimally invasive procedure is a ductoscopy within the mammary ducts of breast tissue. A micro-endoscope can be advanced through the mammary ducts providing endoscopic visualization of cells and tissue within the ducts. If a suspected tissue site is located, a biopsy can be performed intraductally. However, the amount of tissue that can be excised using an intraductal procedure can be limited due to the size of the instruments involved. The physician may choose not to perform a biopsy at that time, but rather perform a subsequent ductoscopy at a later time to observe whether any changes in the suspect tissue have occurred. Due to physical changes that can occur within the ducts and the multiple ductal branching and tissue between the two ductoscopies, the physician may be unable to definitively locate the suspect tissue site a second time.

### SUMMARY

A system and method for marking a tissue site are described. In general, in one aspect, the invention features a method for marking a tissue site internally in a patient's body. An internal tissue site is identified using an internal imaging device. A marker is deployed into or near the tissue site, where the marker can be later located using an imaging device external to the patient's body. After withdrawing the internal imaging device, the location of the marker is identified using an external imaging device. A biopsy of the internal tissue site is performed based on the location of the marker.

Implementations of the invention may include one or more of the following features. The internal imaging device can be a micro-endoscope and the internal tissue site can be located within a patient's duct. Deploying a marker into or near the tissue site can include: inserting a needle loaded with the marker at a distal end of the needle into a working channel within the micro-endoscope; advancing the distal end of the needle to the internal tissue site; and, while maintaining a position of the marker relative to the internal tissue site, withdrawing the needle relative to the marker thereby deploying the marker into the internal tissue site. The marker can include one or more self-expanding components and deploying the marker can include releasing the marker from within the needle such that the one or more self-expanding components expand from a compacted position into an expanded position.

In general, in another aspect, the invention features a system for marking a tissue site internally in a patient's body. The system includes an internal imaging device, a marker and a deployment system. The internal imaging device is configured to assist a user in visualizing an internal tissue site. The marker is configured to embed within or near the tissue site and is configured to be visible by an imaging device external to the patient's body. The deployment system is configured to deploy the marker to the tissue site.

Implementations of the invention may include one or more of the following features. The internal imaging device can be a micro-endoscope and the internal tissue site can be located within a duct. The marker can include one or more self-expanding components such that when the marker is deployed by the deployment system, the one or more self-expanding components expand from a compacted position into an expanded position. The marker can include a core having a first end and second end, and the one or more self-expanding components can include one or more barbs located at each of the first and second ends of the core. The marker can include four barbs located at the first end of the core spaced approximately 90° from one another, and four barbs located at the second end of the core spaced approximately 90° from one another. The four barbs located at the first end of the core can be offset from the four barbs located at the second end of the core by approximately 45°. The one or more barbs can project radially inwards toward a center of the core and expand above the core. Alternatively, the one or more barbs can project radially outwards away from a center of the core.

In another implementation, the one or more barbs can project from the first and second ends of the core approximately orthogonally relative to a longitudinal axis of the core. The core can be formed at least partially from a radiopague material and/or at least partially from a Nitinol tube. The marker can include a core formed from a radiopaque material and the one or more self-expanding components can include one or more longitudinal wings located along the core. The marker can include a core formed from a radiopaque material and one or more fixed barbs extending outwardly from the core. The marker can include a core formed from a radiopaque material and one or more raised ridges formed along an exterior surface of the core. The one or more raised ridges can be a plurality of mono-directional ridges or a plurality of bi-directional ridges. The marker can include a core formed from a radiopaque material and have a dumb-bell shape, which may or may not include one or more barbs projecting from a first end and a second end of the core. The marker can include an expandable core formed at least partially from a radiopaque material. The expandable core can include an expandable braid formed at least partially from Nitinol wire. In another implementation, the expandable core can include an expandable stent formed at least partially from Nitinol wire. The marker can include a first end having a corkscrew configuration and a second end configured to detachably connect to a shaft.

In one implementation, the deployment system included in the system is a needle and the marker is an injectable radiopaque material, e.g., a biocompatible epoxy. In another implementation, the marker is a shape set wire configured to ball up upon release from the deployment mechanism, e.g., the shape set wire can be formed from Nitinol. In another implementation, the marker can include a first component that embeds at or near the tissue site and a second component providing a trail to the tissue site. For example, the second component can be a guidewire or a biocompatible ink.

In one implementation, the internal imaging device includes a working channel and the deployment system can be positioned within the working channel of the internal imaging device.

The deployment mechanism can include: a housing having an interior cavity; a handle extending from the interior cavity of the housing and slidably moveable relative to the housing; a first locking mechanism configured to lock the handle in position relative to the housing; a needle positioned within a lumen of the handle and protruding from a distal end of the housing; a push rod positioned within a lumen of the needle; a second locking mechanism configured to lock the push rod in a position; and a needle mover mechanism configured to move the needle relative to the handle and the push rod.

Implementations of the invention can realize one or more of the following advantages. Tissue that may be a candidate for a future external biopsy that is identified during a ductoscopy can be marked to facilitate identification during the biopsy. A trail to the candidate tissue can be left within the duct, to facilitate locating the tissue during a subsequent ductoscopy under endoscopic visualization. For example a guidewire attached to a marker left at the tissue site, or an ink trail leading to the tissue site, can be used.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a flowchart showing a process for marking a tissue site.

FIG. 2 shows a micro-endoscope system.

FIG. 3 shows a cross-section of a micro-endoscope shown in FIG. 2 taken along line 3-3.

FIG. 4 shows an embodiment of a micro-endoscope including an introducer sheath.

FIG. 5 shows an embodiment of a marker.

FIG. 6 shows a marker deployment system.

FIG. 7 is a flowchart showing a process for intraductal marking of a tissue site.

FIGS. 8-18C show alternate embodiments of a marker.

FIGS. 19 and 20 show cross-sectional views of alternate embodiments of a micro-endoscope.

Like reference symbols in the various drawings indicate like elements.

### DETAILED DESCRIPTION

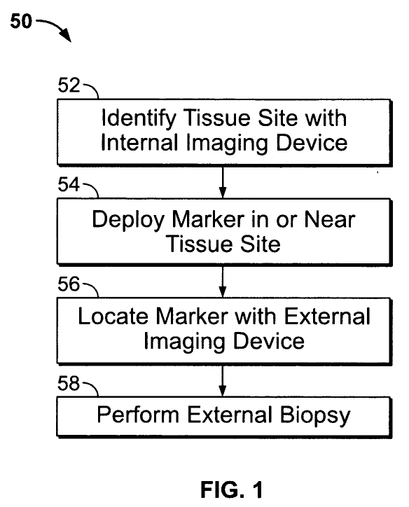

A method and system for marking a tissue site within a patient's body are described. Referring to FIG. 1, a process 50 for marking a tissue site is shown. An internal tissue site can be identified using an internal imaging device (Step 52). A marker can be deployed into or near the tissue site (Step 54). The marker can be later located using an imaging device external to the patient's body (Step 56). If desired, a biopsy can then be performed externally at the site identified by the marker to excise a sufficient amount of tissue to provide a suitable biopsy sample (Step 58).

In one implementation, the internal imaging device can be a micro-endoscope for performing a ductoscopy. For example, mammary ductoscopy is a procedure that uses a micro-endoscope including a tiny scope with a lens to look inside the milk ducts of the breast. Abnormalities can be observed and changes in the cell lining monitored.

Although preferably, a biopsy sample is taken intraductally under direct endoscopic visualization, it may not be possible to excise enough tissue sample under such conditions to perform a definitive diagnosis. Accordingly, a marker is placed into or near a suspect site (i.e., a site for a potential subsequent biopsy) under endoscopic visualization. An external biopsy can then be performed using an external imaging device for guidance, e.g., ultrasound or fluoroscopy, by locating the marker, and therefore the suspect site, and extracting enough tissue volume to improve the chances of making a definitive diagnosis.

The technique can be implemented as follows. If a suspect tissue site is observed during the ductoscopy, i.e., a site including a tissue mass that is a candidate for a biopsy, then a marker is introduced into or near the suspect site. In one implementation, once the micro-endoscope is inside a duct and the suspect site has been observed, a marker deployment device can be advanced down a working channel of the micro-endoscope. Under endoscopic visualization, the marker deployment device can be advanced into or near the suspect site and the marker deployed into a location in or near the suspect site. The marker is configured so as to be detectable by an imaging device external to the patient's body, e.g., using an ultrasound transducer or by fluoroscopy. By detecting the marker, the suspect site identified by ductoscopy can be located and an external biopsy performed to extract tissue from the suspect site.

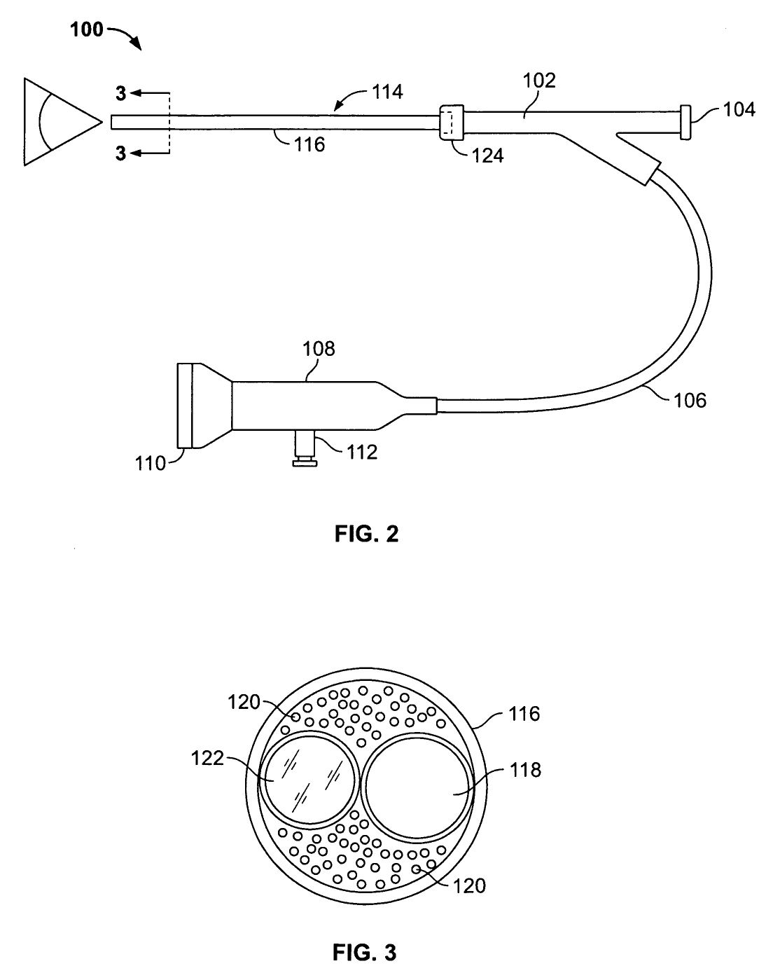

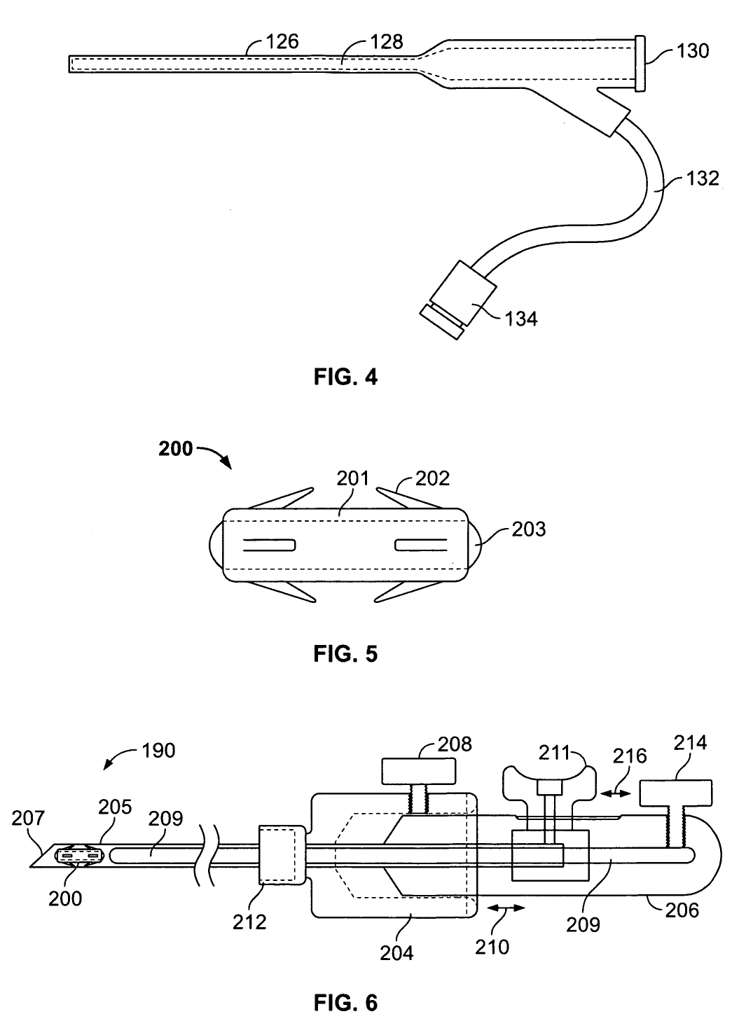

FIGS. 2 and 3 show one implementation of a micro-endoscope system 100 including a working channel. A proximal end of a Y-connector 102 includes a working channel inlet 104 and connects to a tube 106 leading to an endoscope housing 108. The tube 106 can protect an image guide and illumination fibers. The endoscope housing 108 can include an eyecup 110 and a light post 112. The light post 112 (or connector) allows the micro-endoscope 114 to connect to a light source, e.g., by a cable, thereby transmitting light from the light source to the distal end of the micro-endoscope 114 to illuminate the field of vision. A distal end of the Y-connector 102 can connect to a micro-endoscope 114.

FIG. 3 shows a cross-sectional view of the micro-endoscope 114 taken along line 3-3. The micro-endoscope 114 includes an outer sheath tubing 116, the working channel 118, illumination fibers 120, and an objective lens 122. The outer sheath tubing 116 can be made from stainless steel, plastic or another suitable material. A luer adapter 124 can connect the Y-connector 102 to the micro-endoscope 114.

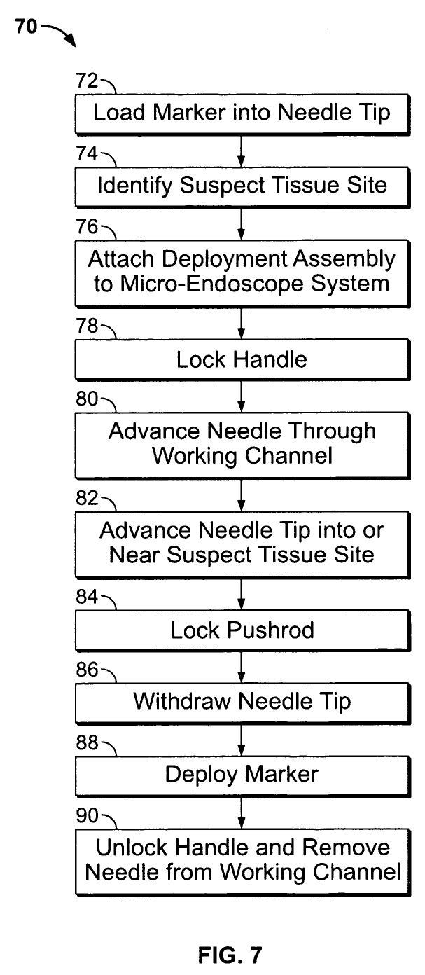

Referring to FIG. 4, in one embodiment, the micro-endoscope 114 can be configured to be introduced into ducts by an introducer sheath 126. The introducer sheath 126 can be chosen to accommodate entry into the mammary ducts by the natural openings and/or orifices of a patient's nipple. For example, the outer diameter can be in a range of approximately 0.35 mm to 1.5 mm. In one embodiment, the outer diameter is in the range of approximately 0.9 to 1.1 mm. The outer diameter of the micro-endoscope 114 shown in FIG. 1 can be slightly smaller than the inner diameter of the introducer sheath 126, thereby maximizing the inner diameter of the working channel 118. To further maximize the inner diameter of the working channel 118, the outer diameter of the micro-endoscope 114 can be maximized, and thus the inner diameter of the introducer sheath 126 maximized. In one implementation, the outer diameter of the micro-endoscope 114 can be in the range of 0.3mm to 1.3mm.

The introducer sheath 126 includes a lumen 128 for receiving the micro-endoscope 114 through an inlet end 130. Optionally, the introducer sheath 126 can include tubing, e.g., PVC tubing having a connector 134, e.g., a luer connector, to connect to an irrigation fluid source. Irrigation fluid can thereby be pumped through the introducer sheath 126 to provide irrigation of the duct during, before or after the ductoscopy, as required.

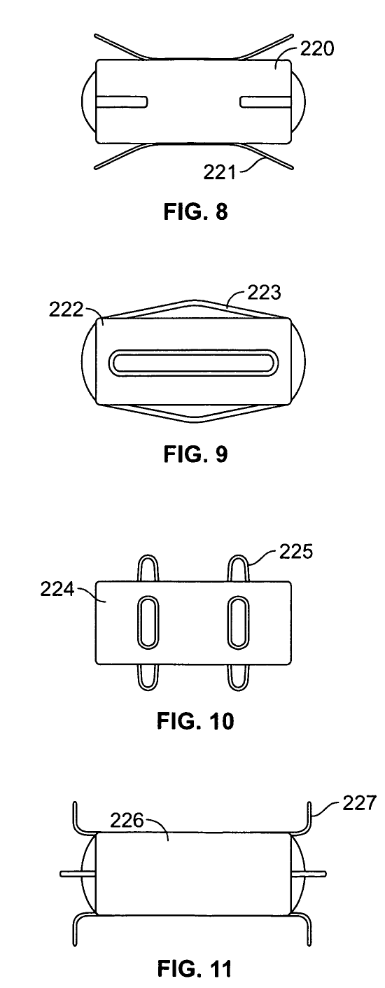

One implementation of a marker 200 is shown in FIG. 5. The marker includes a tubular core 201 and barbs 202 on either end. In this implementation, the barbs are spaced approximately 90° from one another along the circumference of the core 201 and project radially inwards toward the center of the core 201, and extend away from and above the outer diameter center of the core 201. The barbs 202 can be laser cut and then shape set to a desired size and angle of protrusion. A memory material, such as Nitinol or Elgiloy, can be used, such that the barbs 202 can be in a compacted position prior to deployment and self-expand to the expanded position shown upon deployment. The core 201 or at least a portion of the core, for example, wire 203, can be formed from a radiopaque material, e.g., Platinum, to enhance the visibility of the marker 200 under fluoroscopy.

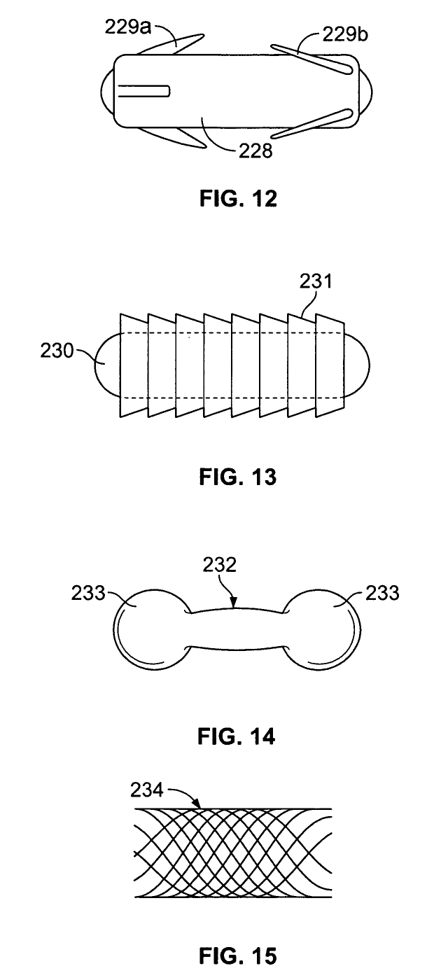

Referring to FIG. 6, a marker deployment handle and catheter assembly 190 is shown. The assembly 190 includes a handle 206 slidably moveable within a housing 204. A connector 212 on one end of the housing 204 is configured to mate with a connector on a working channel of an endoscope, for example, a connector positioned at working channel inlet 104 of the micro-endoscope system 100 shown in FIG. 2. A needle 205 extends from the housing 204 and is configured to fit within the working channel of an endoscope, e.g., micro-endoscope 114. The needle 205 includes a sharp distal tip 207 configured to pierce tissue. A pushrod 209 is positioned and is moveable within a lumen of the needle 205. A locking mechanism 214 can be activated to lock the pushrod 209 into a position, while leaving the needle 205 free to move. A second locking mechanism 208 can be activated to lock the handle 206 into a position. When not activated, the handle 206 is slidably movable within the housing 204 in the direction of arrow 210. In the implementation shown, the locking mechanisms 208 and 214 are screws, although in other implementations, other locking mechanisms can be used.

When the handle 206 and pushrod 209 are locked into a position, a needle mover 211 can be activated to move the needle relative to the pushrod 209 and handle 206 in the direction of arrow 216. In the implementation shown, the needle mover 211 is a thumb slide device that slides the needle 215, although in other implementations, other configurations of needle mover 211, e.g., a threaded configuration, can be used.

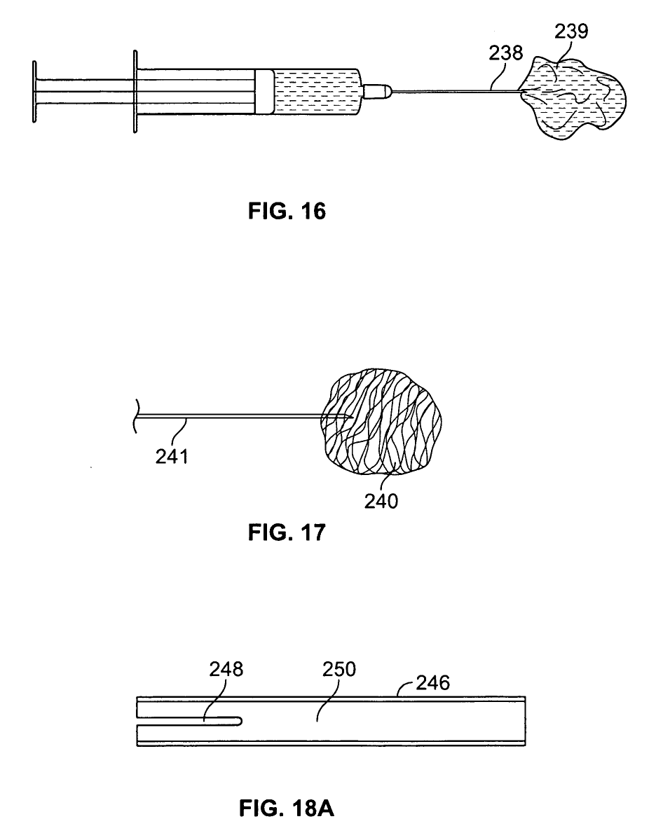

Referring to FIG. 7, a process 70 for embedding a marker into a tissue site within a duct in conjunction with a ductoscopy is shown. The marker 200 is loaded into the needle tip 207 (Step 72). Once a suspect site is identified under endoscopic visualization (Step 74), the marker deployment handle and catheter assembly 190 is attached to the micro-endoscope system 100 (Step 76). The assembly 190 is slid into working channel inlet 104 of the micro-endoscope system 100 and connected to the system 100, for example, by a swivel luer connector 212 connecting to a luer hub on the working channel inlet 104 of the system 100. In one implementation, the length of the needle 205 is chosen such that when the connection between the outer housing 204 and the working channel inlet 104 is made, the needle tip 207 is just slightly recessed inside the working channel 118 of the micro-endoscope 114.

The needle 205 is advanced through the working channel 118 of the micro-endoscope 114 (Step 78). The handle 206 is locked to the Y-connector 102 using the locking mechanism 208 when the needle 205 is inserted into the working channel 118 (Step 80), which allows the physician to move the needle tip 207 by sliding the handle 206 forward. Under endoscopic visualization, e.g., fiberoptics, the needle tip 207 is advanced into or near the suspect site (Step 82). In one implementation, depth markers, e.g., on the needle 205 or on the handle 206, can guide the insertion depth. With the needle tip 207 in the desired location at or near the suspect site, the push rod 209 is locked into place using the locking mechanism 214 (Step 84). A lever or slide (e.g., thumbslide 211) is then activated to withdraw the needle tip 207 relative to the marker 200, which is held in place by the locked pushrod 209 (Step 86). Once the marker 200 is exposed, i.e., released from the needle tip 207, the marker is deployed into the tissue site (Step 88). In the implementation shown of the marker 200, the barbs 202 self-expand upon deployment and fix the marker 200 within the surrounding tissue.

The deployment handle 206 can then be unlocked and the needle 205 removed from the working channel 118 (Step 90). Optionally, an external biopsy can be performed, e.g., under fluoroscopic or ultrasonic guidance, by locating the marker 200 and excising tissue at the suspect site as identified by the marker 200. The marker 200 can be removed with the tissue excised at the time of the biopsy.

In one implementation, a “bread crumb” trail can be left to the suspected site. For example, a guide wire, attached to or separate from the marker 200, can be left in the patient's duct to mark the trail from the duct inlet to the suspect tissue site. In another implementation, a permanent biocompatible ink is used to mark the trail within the duct to the suspect tissue site. The guidewire or ink trail allows a physician to return to the suspect site for future follow-up during a subsequent ductoscopy.

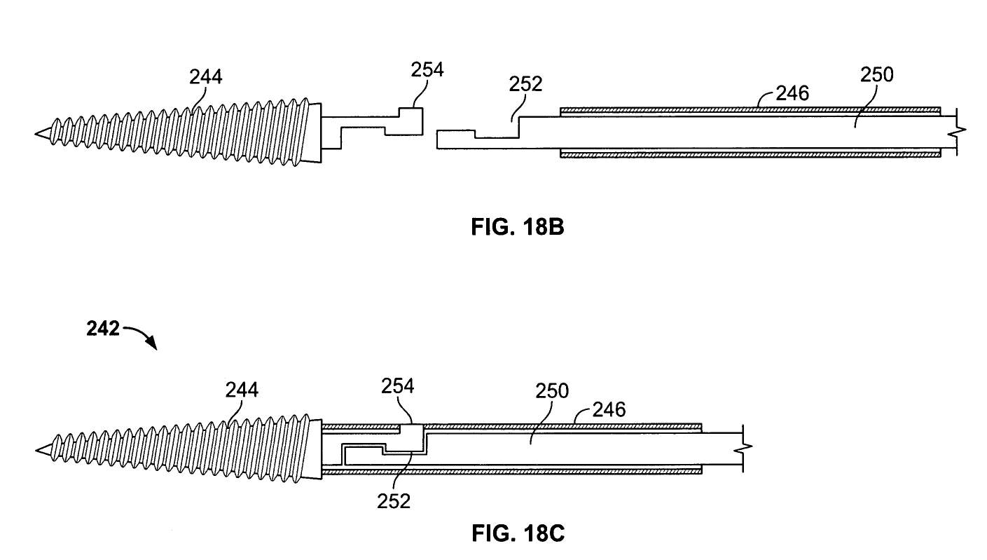

The marker 200 described above is illustrative-other configurations of marker can be used. Some alternative embodiments of the marker 200 are shown in FIGS. 8-18. FIG. 8 shows a marker including a core 220 and self-expanding barbs 221 located at the ends of the core 220. The barbs 221 project radially away from the center of the core 220. The core 220 can be formed from a laser cut tube and made at least partially from a radiopaque material, e.g., platinum. The marker shown includes two barbs 221 per end, however, there can be any number of barbs per end (e.g., 1-8). In one implementation, the barbs 221 are formed from a memory shape material, e.g., Nitinol, or a metal such as Elgiloy, a chromium-nickel-alloy. The core 220 may also be formed in whole or in part from the same type of material.

FIG. 9 shows a marker including a core 222 and longitudinal wings 223. The core 222 can be made at least partially from a radiopaque material, e.g., platinum. The longitudinal wings 223 can be self-expanding, i.e., expandable from a compacted position to the expanded position shown. In one implementation, the wings 223 are formed from a memory shape material, e.g., Nitinol or a metal, e.g., Elgiloy.

FIG. 10 shows a marker including raised, fixed barbs 225 protruding from a core 224. The core can be made at least partially from a radiopaque material, e.g., platinum. The barbs 225 can be made from any suitable material including, for example, stainless steel, Nitinol or Elgiloy. In one implementation, the barbs 225 can be formed as “spring leafs” that can be flush with the outer diameter of the core 224 when inside the needle 205 and can spring open after deployment into the patient's tissue.

FIG. 11 shows a marker including a core 226 and self-expanding barbs 227 positioned at the ends of the core 226. The self-expanding barbs 227 project approximately orthogonally relative to the longitudinal axis of the core 226. The core 226 can be made at least partially from a radiopaque material, e.g., platinum. In one implementation, the barbs 227 can be made from a memory shape material, e.g., Nitinol or a metal, e.g., Elgiloy.

FIG. 12 shows a marker similar to the marker 200 described above in reference to FIG. 5, except the barbs 229a-b located on the opposite ends of the core 228 are offset from one another by approximately 45°. That is, the four barbs 229a on a first end are spaced at approximately 90° from one another about the circumference of the core 228, and the four barbs 229b on the opposite end (the second end) are also spaced at approximately 90° from one another, but offset approximately 45° from the barbs 229a on the first end. This configuration may improve migration resistance within the tissue. More or fewer barbs per end can be used.

FIG. 13 shows a marker having a core 230 and raised ridges 231 protruding from the core 230. The raised ridges 231 may be mono-directional (as shown) or bi-directional. The raised ridges may be threads. The core 230 can be made at least partially from a radiopaque material, e.g., platinum. In one implementation, the core 230 is machined to form the ridges and is made from a material such as stainless steel, Nitinol or Elgiloy.

FIG. 14 shows a marker having a profile similar to a “dumb-bell”. The marker includes a core 232 and bulbous shaped ends 233. In one implementation, barbs can be included on the ends 233. At least a portion of the marker can be formed from a radiopaque material, e.g., platinum.

FIG. 15 shows a marker 234 in the form of an expandable braid or stent. The marker 234 is formed from wires. In one implementation, the wires can be formed from a memory shape material, e.g., Nitinol or a metal, e.g., Elgiloy, and additionally at least some of the marker 234 can be formed from a radiopaque material, e.g., platinum. The ends of the marker 234 can be open, such that the wire ends help secure the device in or near a tissue site.

FIG. 16 shows an injectable, radiopaque marker 239. The marker 239 is deployed from a needle tip 238 into or near a suspect tissue site. In one implementation, the marker 239 is a biocompatible epoxy with radiopacity.

FIG. 17 shows a marker 240 formed from a shape set wire or coil (e.g., Nitinol or Elgiloy) that is set to ball-up upon release from a deployment mechanism 241.

FIGS. 18A-C show one implementation of a marker and deployment system 242 configured with a corkscrew marker 244 positioned at an end of a deployment shaft 246. FIG. 18A shows a top view of the shaft 246. In this implementation, the shaft is torquable and includes a first key way 248. FIG. 18B shows a side view of the corkscrew marker 244 and the shaft 246. A capture rod 250 is included within the shaft 246 and includes a second key way 252 is shown. A tab 254 included on a proximal end of the corkscrew marker 244 is configured to align with the second key way 252 formed in the capture rod 250 and with the first key way 248 formed in the shaft. Accordingly, the corkscrew marker 244 turns with the shaft 246. FIG. 18C shows a cross-sectional side view of the system 242. When the corkscrew marker 244 is aligned with the first and second key ways 248, 252 within the shaft 246, the marker 244 cannot be separated from the deployment shaft 246.

To deploy the marker 244, the physician turns the shaft 246 and capture rod 250 simultaneously with a handle attached to a proximal end of the shaft 246. Once the marker 244 is positioned with the tissue site, the handle can be used to slide the shaft 246 to uncover the keyed joint between the marker 244 and the capture rod 250. The marker 244 can thereby be released from the capture rod 250, e.g., by rotating the rod 250. The shaft 246 and capture road 250 can then be removed from the patient.

The above described markers are some examples of configurations of markers that can be embedded in or near a suspected tissue site. Other configurations can be used, including combinations of two or more of the above described implementations.

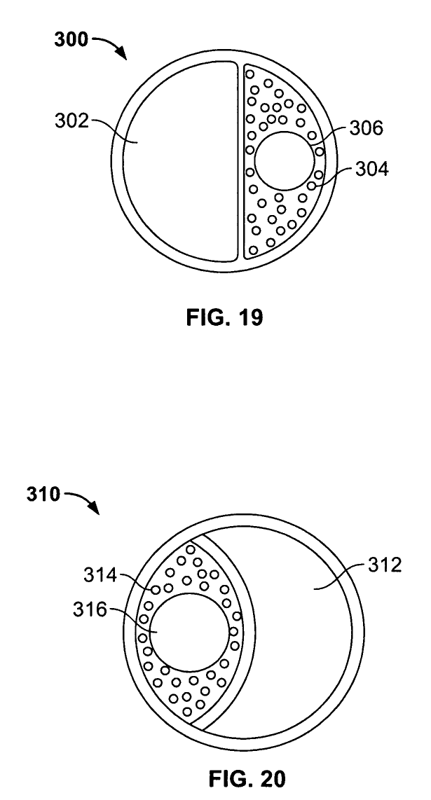

Referring again to FIG. 3, a cross-sectional view of one implementation of a micro-endoscope 114 is shown. The micro-endoscope 114 can have other configurations. A cross-sectional view of an alternative micro-endoscope is shown in FIG. 19. The micro-endoscope 300 includes an approximately D-shaped working channel 302, illumination fibers 304 and an objective lens/image guide 306. A cross-sectional view of another alternative micro-endoscope 310 is shown in FIG. 20. The micro-endoscope 310 includes an approximately crescent-shaped working channel 312, illumination fibers 314 and an objective lens/image guide 316.

The method and system for internally marking a tissue site with a marker that can be located with an external imaging device for a subsequent external biopsy has been described for illustrative purposes in the context of an intraductal tissue site identified by a ductoscopy. However, the method and system described herein can be implemented in other contexts where an internal imaging device is used to locate the tissue site, the marker is introduced internally and can be visualized using an external imaging device. The intraductal system described herein is exemplary and not limiting.

A number of embodiments of the invention have been described. Nevertheless, it will be understood that various modifications may be made without departing from the spirit and scope of the invention. For example, the steps set forth in FIGS. 1 and 7 can be performed in a different order and still achieve the desired results.

What is claimed is:

1. A method for marking a tissue site internally in a patient's body, comprising:
identifying an internal tissue site using an internal imaging device; and
deploying a marker into or near the tissue site, where the marker can be later located using an imaging device external to the patient's body.

2. The method of claim 1, further comprising:
after withdrawing the internal imaging device, identifying the location of the marker using an external imaging device; and
performing a biopsy of the internal tissue site based on the location of the marker.

3. The method of claim 2, wherein the internal imaging device is a micro-endoscope and the internal tissue site is located within a patient's duct.

4. The method of claim 3, wherein deploying a marker into or near the tissue site comprises:
inserting a needle loaded with the marker at a distal end of the needle into a working channel within the micro-endoscope;
advancing the distal end of the needle to the internal tissue site; and
while maintaining a position of the marker relative to the internal tissue site, withdrawing the needle relative to the marker thereby deploying the marker into the internal tissue site.

5. The method of claim 4, wherein the marker includes one or more self-expanding components and wherein deploying the marker comprises releasing the marker from within the needle such that the one or more self-expanding components expand from a compacted position into an expanded position.

6. A system for marking a tissue site internally in a patient's body, comprising:
an internal imaging device configured to assist a user in visualizing an internal tissue site;
a marker configured to embed within or near the tissue site, where the marker is configured to be visible by an imaging device external to the patient's body; and
a deployment system configured to deploy the marker to the tissue site.

7. The system of claim 6, wherein the internal imaging device is a micro-endoscope and the internal tissue site is located within a duct.

8. The system of claim 6, wherein the marker includes one or more self-expanding components such that when the marker is deployed by the deployment system, the one or more self-expanding components expand from a compacted position into an expanded position.

9. The system of claim 8, wherein the marker comprises:
a core having a first end and second end; and
the one or more self-expanding components comprise one or more barbs located at each of the first and second ends of the core.

10. The system of claim 9, wherein the marker comprises:
four barbs located at the first end of the core spaced approximately 90° from one another; and
four barbs located at the second end of the core spaced approximately 90° from one another.

11. The system of claim 10, wherein the four barbs located at the first end of the core are offset from the four barbs located at the second end of the core by approximately 45°.

12. The system of claim 9, wherein the one or more barbs project radially inwards toward a center of the core and expand above the core.

13. The system of claim 9, wherein the one or more barbs project radially outwards away from a center of the core.

14. The system of claim 9, wherein the one or more barbs project from the first and second ends of the core and are approximately orthogonal relative to a longitudinal axis of the core.

15. The system of claim 9, wherein core is formed at least partially from a radiopague material.

16. The system of claim 9, wherein the core comprises a Nitinol tube.

17. The system of claim 8, wherein the marker comprises:
a core formed from a radiopaque material; and
the one or more self-expanding components comprise one or more longitudinal wings located along the core.

18. The system of claim 6, wherein the marker comprises:
a core formed from a radiopaque material; and
one or more fixed barbs extending outwardly from the core.

19. The system of claim 6, wherein the marker comprises:
a core formed from a radiopaque material; and
one or more raised ridges formed along an exterior surface of the core.

20. The system of claim 19, wherein the one or more raised ridges comprise a plurality of mono-directional ridges.

21. The system of claim 19, wherein the one or more raised ridges comprise a plurality of bi-directional ridges.

22. The system of claim 6, wherein the marker comprises:
a core formed from a radiopaque material and having a dumb-bell shape.

23. The system of claim 22, wherein the marker further comprises:
one or more barbs projecting from a first end and a second end of the core.

24. The system of claim 6, wherein the marker comprises:
an expandable core formed at least partially from a radiopaque material.

25. The system of claim 24, wherein the expandable core comprises an expandable braid formed at least partially from Nitinol wire.

26. The system of claim 24, wherein the expandable core comprises an expandable stent formed at least partially from Nitinol wire.

27. The system of claim 6, wherein the marker comprises:
a first end having a corkscrew configuration and a second end configured to detachably attach to a deployment shaft.

28. The system of claim 6, wherein:
the deployment system comprises a needle; and
the marker comprises an injectable radiopaque material.

29. The system of claim 28, wherein the injectable radiopaque material comprises a biocompatible epoxy.

30. The system of claim 6, wherein the marker comprises a shape set wire configured to ball up upon release from the deployment mechanism.

31. The system of claim 30, wherein the shape set wire is formed from Nitinol.

32. The system of claim 6, wherein the marker includes a first component that embeds at or near the tissue site and a second component providing a trail to the tissue site.

33. The system of claim 32, wherein the second component comprises a guidewire.

34. The system of claim 32, wherein the second component comprises a biocompatible ink.

35. The system of claim 6, wherein:
the internal imaging device includes a working channel; and
the deployment system can be positioned within the working channel of the internal imaging device.

36. The system of claim 6, wherein the deployment mechanism comprises:
a housing including an interior cavity;
a handle extending from the interior cavity of the housing and slidably moveable relative to the housing;
a first locking mechanism configured to lock the handle in position relative to the housing;
a needle positioned within a lumen of the handle and protruding from a distal end of the housing;
a push rod positioned within a lumen of the needle;
a second locking mechanism configured to lock the push rod in a position; and
a needle mover mechanism configured to move the needle relative to the handle and the push rod.
